(19) United States
(12) Patent Application Publication
(10) Pub. No.: US 2014/0058486 A1
(43) Pub. Date: Feb. 27, 2014

(54) SECURABLE ICE PACK DEVICE

(71) Applicant: Wardell Moore, JR., Chicago, IL (US)

(72) Inventor: Wardell Moore, JR., Chicago, IL (US)

(21) Appl. No.: 13/975,960

(22) Filed: Aug. 26, 2013

Related U.S. Application Data
(60) Provisional application No. 61/693,098, filed on Aug. 24, 2012.

(51) Int. Cl. A61F 7/10

(52) U.S. Cl. CPC A61F 7/10
USPC 607/112

(57) ABSTRACT

An ice pack device that may be secured to an appendage to be treated. The ice pack device may include a body portion that includes an ice pack compartment. The ice pack compartment may secure ice, refrigerant gel, or liquids. Extending from the body portion may be at least one first strap and at least one second strap. The ice pack device may be secured to the appendage by wrapping the straps around the appendage and releasably connecting them.

### CROSS-REFERENCE TO RELATED APPLICATION

This application claims the benefit of priority of U.S. provisional application No. 61/693,098, filed Aug. 24, 2012, the contents of which are herein incorporated by reference.

### BACKGROUND OF THE INVENTION

The present invention relates to an ice pack and, more particularly, to and an ice pack that may be secured to an appendage.

An ice pack or gel pack is a plastic sac of ice, refrigerant gel, or liquid. The simplest type of ice pack is simply a sack, bag or towel filled with cubed or crushed ice. Ice packs are commonly used as a cold compress to alleviate the pain or reduce swelling of minor injuries. However, currently a user must hold the ice pack against the injured areas, which may be inconvenient.

As can be seen, there is a need for an ice pack that may be secured to an appendage.

### SUMMARY OF THE INVENTION

In one aspect of the present invention, an ice pack device comprises: a body portion comprising a first side, a second side, a top end, a bottom end, a front surface, and a rear surface wherein the body portion comprises an ice pack compartment; at least one first strap extending from the first side and at least one second strap extending from the second side; and a connector releasably connecting the first strap and the second strap.

In another aspect of the present invention, a method of applying ice to an appendage comprises: providing an ice pack device comprising: a body portion comprising a first side, a second side, a front surface and a rear surface, wherein the body portion comprises an ice pack compartment comprising ice; and at least one first strap extending from the first side and at least one second strap extending from the second side; placing the rear surface of the ice pack device against the appendage to be treated; wrapping the first strap and the second strap around the appendage to be treated; and connecting the first strap and the second strap together, thereby securing the ice pack device to the appendage.

These and other features, aspects and advantages of the present invention will become better understood with reference to the following drawings, description and claims.

### BRIEF DESCRIPTION OF THE DRAWINGS

FIG. 1 is a perspective view of the present invention;

FIG. 2 is a cross-sectional view taken along line 2-2 of FIG. 1; and

FIG. 3 is a perspective view showing the present invention in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an ice pack device that may be secured to an appendage to be treated. The ice pack device may include a body portion that includes an ice pack compartment. The ice pack compartment may secure ice, refrigerant gel, or liquids. Extending from the body portion may be at least one first strap and at least one second strap. The ice pack device may be secured to the appendage by wrapping the straps around the appendage and releasably connecting them.

The present invention may include an ice pack that may be strapped to an appendage. For example, the present invention may be strapped to a user's knee and the user may still remain active with the ice pack on. In certain embodiments, the present invention may be made of a flexible material such as spandex and may include Velcro® straps and an ice compartment made of a polymer, such as silicon. Therefore, the present invention may be a one piece device that holds the silicon ice compartment in place on the knee using the Velcro® straps that may wrap around and secure the present invention.

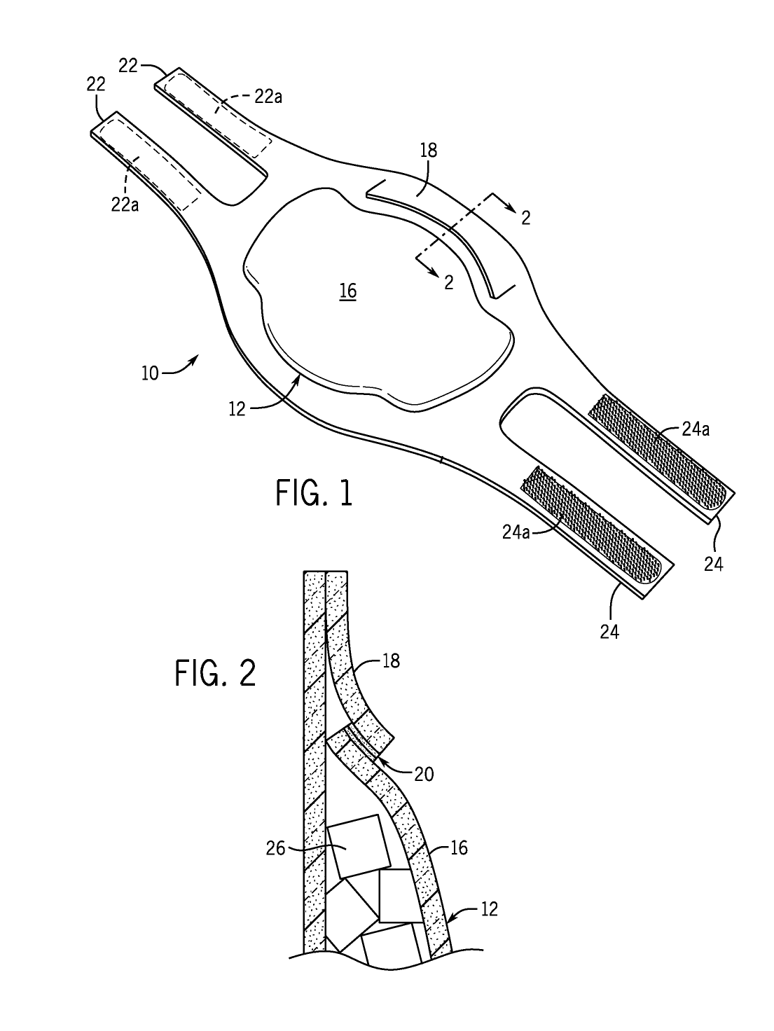

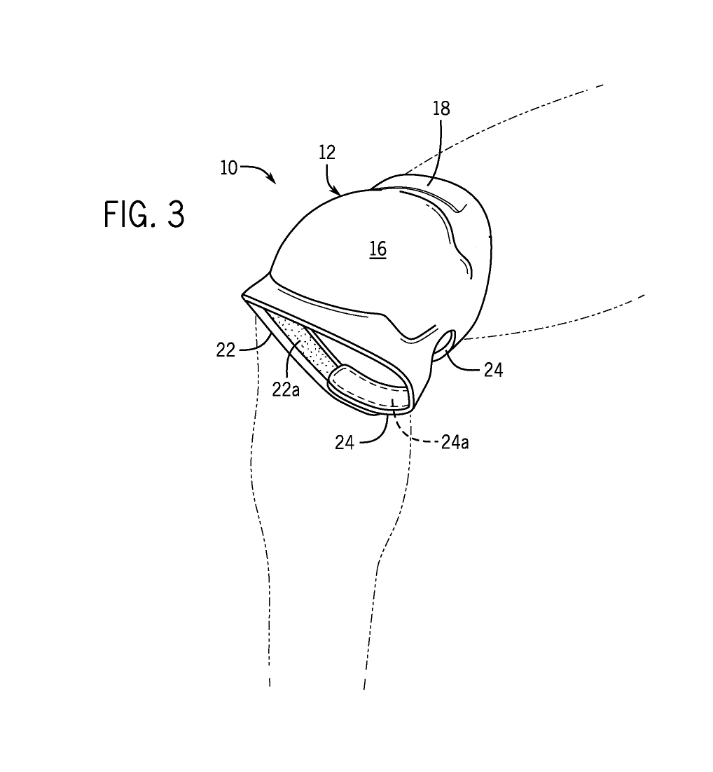

Referring to FIGS. 1 through 3, the present invention may include an ice pack device 10. The ice pack device 10 may include a body portion 12 having a first side, a second side, a top end, a bottom end, a front surface, and a rear surface. The body portion 12 may include an ice pack compartment 16. The present invention may further include at least one first strap 22 extending from the first side and at least one second strap 24 extending from the second side. The present invention may further include a connector 22a, 24a that may releasably connect the first strap 22 and the second strap 24 together.

In certain embodiments of the present invention, the at least one first strap 22 may include two first straps 22, and the at least one second strap 24 may include two second straps 22. The connector 22a, 24a of the present invention may include a hook and loop fastener, a clip, a button, a snap button, a coupling, and the like. As illustrated in the Figures, the connector 22a, 24a may include a hook component 22a on the rear surface of the first straps 22 and a loop component 24a on the front surface of the second straps 24. Therefore, the first straps 22 may be releasably connectable with the second straps 24. In certain embodiments, the connecter 22a, 24a and straps 22, 24 may be adjustable in length to accommodate different sized individuals.

As mentioned above, the ice pack device 10 may include an ice compartment 16. The ice compartment 16 may be made of a water proof material, such as a polymer, to contain the ice and water within. For example, the ice compartment 16 may be made of silicon. The ice compartment 16 may contain ice 26, refrigerant gel, or liquid. In certain embodiments, the ice compartment 16 may form an opening 18 near the top end of the body 12. Therefore, a user may place ice 26 within the ice compartment 16 through the opening 18. In certain embodiments, a connector 20 may be used to open and close the opening. The connector 20 may be any connector mentioned above, such as the hook and loop fastener.

A method of applying ice to an appendage may include the following. The ice pack device mentioned above may be provided. If ice is used, the user may fill the ice compartment with ice using the opening. If a refrigerant gel is used, the user may place the ice pack device within the freezer for an appropriate amount of time for the gel to freeze. The user may place the rear surface of the ice pack device against the appendage to be treated, such as a knee. The user may wrap the first strap and the second strap around the appendage to connect the first strap and the second strap. The user may tie the first and second strap together or use a connector, such as a hook and loop fastener to connect the first strap and the second strap together, thereby securing the ice pack device to the appendage.

It should be understood, of course, that the foregoing relates to exemplary embodiments of the invention and that modifications may be made without departing from the spirit and scope of the invention as set forth in the following claims.

What is claimed is:

1. An ice pack device comprising:
a body portion comprising a first side, a second side, a top end, a bottom end, a front surface, and a rear surface wherein the body portion comprises an ice pack compartment; and
at least one first strap extending from the first side of the body portion and at least one second strap extending from the second side of the body portion.

2. The ice pack device of claim 1, further comprising a connector releasably connecting the first strap and the second strap.

3. The ice pack device of claim 1, wherein the at least one first strap comprises two straps and the at least one second strap comprises two straps.

4. The ice pack device of claim 2, wherein the connector comprises at least one of a hook and loop fastener, a clip, a button, a snap button, and a coupler.

5. The ice pack device of claim 1, wherein the first strap comprises a hook component on a rear surface and the second strap comprises a loop component on a front surface, wherein the hook and loop components releasably attach.

6. The ice pack device of claim 1, wherein the ice pack compartment forms an opening near the top end and front surface of the body portion, wherein the opening is formed to receive ice.

7. The ice pack device of claim 6, further comprising a connector that opens and closes the opening.

8. The ice pack device of claim 7, wherein the connector is a hook and loop fastener.

9. The ice pack device of claim 1, wherein the ice compartment comprises a refrigerant gel.

10. The ice pack device of claim 1, wherein the ice compartment comprises a cover comprising a polymer.

11. A method of applying ice to an appendage comprising:
providing an ice pack device comprising: a body portion comprising a first side, a second side, a front surface and a rear surface, wherein the body portion comprises an ice pack compartment comprising ice; and at least one first strap extending from the first side and at least one second strap extending from the second side;
placing the rear surface of the ice pack device against the appendage to be treated;
wrapping the first strap and the second strap around the appendage to be treated; and
connecting the first strap and the second strap together, thereby securing the ice pack device to the appendage.
